(19) United States
(12) Patent Application Publication
(10) Pub. No.: US 2012/0245552 A1
(43) Pub. Date: Sep. 27, 2012

(54) Combination Therapy

(75) Inventors: Randice Lisa Altschul, Cliffside Park, NJ (US); Neil David Theise, New York, NY (US); Myron Rapkin, Indianapolis, IN (US); Rebecca O'Brien, Shell Knob, MO (US)

(73) Assignee: POP TEST CORTISOL LLC, Cliffside Park, NJ (US)

(21) Appl. No.: 13/425,964

(22) Filed: Mar. 21, 2012

Related U.S. Application Data
(60) Provisional application No. 61/465,704, filed on Mar. 23, 2011.
(60) Provisional application No. 61/516,035, filed on Mar. 28, 2011.
(60) Provisional application No. 61/516,970, filed on Apr. 11, 2011.

(51) Int. Cl. A61J 1/05; A61P 29/00; A61P 19/00; A61P 19/04; A61P 21/00; A61P 1/00; A61P 1/18; A61P 7/00; A61P 9/00; A61P 5/00; A61P 13/00; A61P 13/12; A61P 13/10; A61P 25/00; A61P 27/00; A61J 1/00; A61K 31/573

(52) U.S. Cl. 604/403; 514/171; 206/438; 206/570

(57) ABSTRACT

This invention relates to compositions and methods for treating GC-responsive conditions and for reducing and preventing side-effects of GC treatment in patients.

### CROSS-REFERENCE TO RELATED APPLICATIONS

This application claims benefit under 35 U.S.C. §119(e) to U.S. Provisional Patent Application 61/465,704 filed Mar. 23, 2011; U.S. Provisional Patent Application 61/516,035 filed Mar. 28, 2011; U.S. Provisional Patent Application 61/516,970 filed Apr. 11, 2011, the disclosures of which are incorporated herein in their entirety.

### BACKGROUND OF THE INVENTION

1. Field of Invention

This invention relates to compositions and methods for treating GC-responsive conditions and for reducing and preventing side-effects of GC treatment in patients.

2. Description of Related Art

The glucocorticoids (GCs) are steroid hormones produced in the adrenal cortex (primarily cortisol, in humans) and synthetically (e.g. prednisone, cortisone, etc). GCs are important steroids for intermediary metabolism, immune, musculoskeletal, connective tissue and brain function. The production and secretion of cortisol is governed by a complex and highly efficient system that includes the hypothalamus, pituitary and the adrenal glands i.e., hypothalamic-pituitary-adrenal axis (HPA). Cortisol secretion has a circadian release rhythm with peak values in early morning and trough values at midnight.

It is known that the hypothalamic-pituitary-adrenal (HPA) axis influences multiple organ systems, maintaining homeostasis in normal conditions and modulating responses to various systemic stresses to restore homeostasis. Cortisol participates in regulation of carbohydrate, protein and fat metabolism. This hormone signals the liver to make glucose and glycogen and the adipose tissues to release lipids and fatty acids into the bloodstream. Similarly, it makes the skeletal muscles release proteins or amino acids into the bloodstream. GCs also regulate the inflammatory response of the body as well, acting, overall, as an immunosuppressant and counterbalance to hyperinflammatory states. All molecules, or ligands, which bind to these various receptors, may have differing kinds of activities at the time of binding. Some ligand binding may lead to activity similar to the normative physiologic response to normal ligands, i.e., agonists. Some ligands may lead to neutral binding and, if these bind more strongly than the physiologic ligand, they may block the normative physiologic response, i.e., antagonists. Some ligands may be variants of steroid hormones derived from chemical alterations to cholesterol. Others may have completely unrelated or novel biochemical structures yet mimic steroid hormone activity.

GCs perform several important functions. These include participating in the regulation of carbohydrate, protein and fat metabolism by signaling the liver to make glucose and glycogen, the adipose tissues to release lipids and fatty acids into the bloodstream, and the skeletal muscles to release proteins or amino acids into the bloodstream. GCs also decrease bone formation.

GCs also regulate the body's inflammatory response as well. GCs are part of the feedback mechanism in the immune system that inhibits immune activity (i.e., inflammation). They cause their effects by binding to the glucorticoid receptor (GCR). The activated GCR complex in turn up-regulates the expression of anti-inflammatory proteins in the nucleus (a process known as transactivation) and represses the expression of pro-inflammatory proteins in the cytosol by preventing the translocation of other transcription factors from the cytosol into the nucleus (transrepression) (Rhen T and Cidlowski J A., NEJM 2005; 353: 1711-23).

Synthetic GCs, such as prednisone (Prelone), triamcinolone (Kenacort), methylprednisolone (Medrol) and dexamethasone (Decadron), are amongst the most potent immunosuppressive and anti-inflammatory drugs available. GCs are widely used in medicine, especially in Rheumatology, Gastroenterology, Pulmonology, Neurology, Dermatology, Oncology, Allergy, (Asthma) and Transplantation and account for over 30 million prescriptions per year in the US alone. They are used to treat conditions that include, but are not limited to autoimmune diseases (e.g., rheumatoid arthritis, inflammatory bowel disease, auto immune dermatitides, Hashimoto's thyroiditis, auto-immune hepatitis), inflammatory and allergic conditions triggered by infection or toxins (e.g., asthma, chronic bronchitis, contact dermatitis) and organ transplant rejection. This anti-inflammatory action may be an appropriate normal modulation of systemic inflammatory responses, but it may also be disease causing under severe environmental or therapeutic stresses in normal individuals or in abnormally responsive individuals when faced with what should be tolerable stressors (e.g., hospitalized elderly).

However, prolonged use of oral GCs at medium or high dose (>7.5 mg/day prednisolone) is hampered by moderate to severe adverse effects, including metabolic changes, water retention potentiating cardiovascular dysfunction, pathological effects in bone (e.g. osteoporosis), skin and muscle, and psychiatric disorders (e.g. depression, anxiety, psychosis). At high doses 35-65% of the patients suffer from severe adverse effects. There is a clear medical need for a compound as efficacious as prednisolone, but safer.

The present invention is a GR-antagonist that selectively blocks GC activities in the tissues resulting in these side effects, while leaving, for example, anti-inflammatory and immune suppressive effects intact, which thus preserves and lengthens the duration of therapeutic utility of a GR-agonist to obtain higher, potentially improved therapeutic dosing, without concern for the limiting side effects of such doses.

The invention relates to 11-(substituted phenyl)-estra-4,9-diene derivatives, a process for the preparation thereof, pharmaceutical compositions containing the same, as well as the use of the derivatives for the manufacture of a medicament and a method of use of GCR (GCR) antagonists to combine with therapeutic agonists of this general receptor class in order to prevent unwanted side effects of therapeutically administered GCR agonists in mammalian subjects (human or other), while preserving or enhancing their therapeutic benefits.

Various 11-(substituted phenyl)-estra-4,9-diene derivatives are known in the art. For example, in German Patent DE 3307143, steroids are described which may carry a variety of substituents at the 11-, 13-, 16- and 17-position. According to DE 3307143, these steroid derivatives have marked affinity to the GC and progesterone receptor and, in addition, they have reasonable affinity to the androgen receptor. Furthermore, in DE 3307143 it is shown that the steroid derivatives have anti-GC activity.

However, Philibert et al. [Agarwal M K (ed): Antihormones in Health and Disease. Front Horm. Res. Basel, Karger, 1991, vol. 19, pp 1-17] discovered that 11-(substituted phenyl)-estra-4,9-diene derivatives disclosed in DE 3307143 are in vivo not very active anti-GC steroids (e.g., the 11-(m-methoxyphenyl)- and 11-(m-methylthiophenyl)- derivatives) or have a relatively high progesterone receptor binding affinity (such as the 11-(p-methoxyphenyl)- and 11-(p-methylthiophenyl)-derivatives). These properties seriously restrict the therapeutic potential of the compounds. Low in vivo activity of the derivatives necessitates the administration of high dosages when they are used in therapy. It is very likely that the incidence of adverse side-effects is thereby increased. Furthermore, high progesterone receptor binding affinity may result in (anti)progestagenic activity, which means that the compound may display more than one (anti)hormonal activity, which limits its clinical use, especially for long-term therapy.

Thus, there is a need for compounds having high GCRbinding affinity and, in addition, high in vivo anti-GC activity, but with other hormonal activities (e.g. androgenic and progestagenic activities) being low.

The present invention relates to combining receptor antagonists and agonists. each with different receptor binding affinities and profiles, to select for the desired agonist activity while preventing undesirable, non-specific agonist activities that limit the possible duration or level of therapeutic dosing of the agonist. Such, complementary, selective receptor antagonists may be identified in several ways:

i. they may be naturally occurring, perhaps found in a different species from the species to be treated with an agonist, and thus have complementary, non-identical binding capacities and activities;

ii. they may be developed through chemical alterations of cholesterol or of physiologically normative steroid hormones (inclusive of, but not limited to the agonist to be selectively antagonized); and/or

iii. they may have structures completely unrelated to cholesterol or other steroid hormones.

Such complementary, selective antagonism may be applied as parallel, but separate dosing with the agonist (particularly if the half life and kinetics of the selective antagonist differs from that of the agonist to be partially opposed) or may be combined as part of a single dose (pill, capsule, infusible liquid, etc.) if the half life and kinetics of the selective antagonist and the agonist are similar or complementary.

All references cited herein are incorporated herein by reference in their entireties.

### BRIEF SUMMARY OF THE INVENTION

The invention provides a composition, comprising: i) a first therapeutic agent which is a GCR agonist or pharmaceutically acceptable salt thereof; ii) a second therapeutic agent which is a GCR antagonist or pharmaceutically acceptable salt thereof; and iii) at least one pharmaceutically acceptable carrier; wherein the GCR agonist and the GCR antagonist are each present in an amount which, in combination, is a therapeutically effective amount for treating a GC-responsive condition in a patient.

The invention further provides a composition of the invention wherein the amount of the GCR antagonist is sufficient to reduce a side-effect of administration of the GCR agonist.

The invention further provides a composition of the invention wherein the GCR agonist is selected from the group consisting of: alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, ulobetasol, and combinations thereof.

The invention further provides a composition of the invention wherein the GCR antagonist is selected from the group consisting of ORG 34517, 11-(substituted phenyl)-estra-4,9-diene derivatives, and 11-(substituted phenyl)-estra-4,9-diene derivatives of formula I

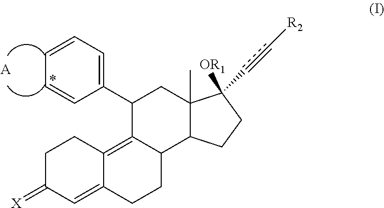

wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond.

The invention further provides a composition of the invention wherein the GCR antagonist is naturally occurring.

The invention further provides a composition of the invention wherein the GCR antagonist is developed through chemical alterations of cholesterol or of physiologically normative steroid hormones.

The invention further provides a composition of the invention wherein the GCR antagonist has a structure unrelated to cholesterol or other steroid hormones.

The invention further provides a composition of the invention wherein the composition is a pharmaceutical composition.

The invention provides a pharmaceutical composition comprising: i) a first therapeutic agent which is a GCR agonist or pharmaceutically acceptable salt thereof; ii) a second therapeutic agent which is a GCR antagonist or pharmaceutically acceptable salt thereof; and iii) at least one pharmaceutically acceptable carrier, wherein the pharmaceutical composition is formulated or manufactured as a liquid, an elixir, an aerosol, a spray, a powder, a tablet, a pill, a capsule, a gel, a geltab, a nano-suspension, a nano-particle, an extended release dosage form, or a topical formulation, further wherein the GCR agonist and the GCR antagonist are each present in an amount which, in combination, is a therapeutically effective amount for treating a GC-responsive condition in a patient.

The invention further provides the pharmaceutical composition of the invention wherein the amount of the GCR antagonist is sufficient to reduce a side-effect of administration of the GCR agonist.

The invention provides a combination therapy which comprises: i) a first therapeutic agent which is a GCR agonist or pharmaceutically acceptable salt thereof;

ii) a second therapeutic agent which is a GCR antagonist or pharmaceutically acceptable salt thereof.

The invention further provides the pharmaceutical composition of the invention which comprises the combination of the invention and a pharmaceutically acceptable carrier.

The invention provides a pharmaceutical composition of the invention made by combining at least one GCR agonist or pharmaceutically acceptable salt thereof, at least one a GCR antagonist or pharmaceutically acceptable salt thereof, and a pharmaceutically acceptable carrier.

The invention provides a pharmaceutical active substance combination comprising: i) a first therapeutic agent which is a GCR agonist or pharmaceutically acceptable salt thereof; ii) a second therapeutic agent which is a GCR antagonist or pharmaceutically acceptable salts thereof, as a combination product for simultaneous, separate, or sequential use.

The invention provides a pharmaceutical dosage form comprising: i) a first therapeutic agent which is a GCR agonist or pharmaceutically acceptable salt thereof; ii) a second therapeutic agent which is a GCR antagonist or pharmaceutically acceptable salt thereof, wherein the first and second agents are in multiple separated dosage units or in a single dosage unit of a combination of the therapeutic agents.

The invention provides a kit for the treatment, amelioration or prevention of a GC-responsive condition in a patient in need of such treatment comprising: (a) the pharmaceutical composition of the invention; and (b) at least one blister package; a lidded blister; a blister card or packet; a clamshell; an intravenous (IV) package, IV packette or IV container; a tray or a shrink wrap comprising the pharmaceutical composition of (a) and instructions for use of the pharmaceutical composition.

The invention provides a product of manufacture comprising: a blister package; a lidded blister; a blister card or packet; a clamshell; an intravenous (IV) package, IV packette or IV container; a tray or a shrink wrap comprising the pharmaceutical composition of the invention and instructions for use of the pharmaceutical composition.

The invention provides a pharmaceutical packaging system comprising: i) a first therapeutic agent which is a GCR agonist, or pharmaceutically acceptable salts thereof; ii) a second therapeutic agent which is a GCR antagonist, or pharmaceutically acceptable salts thereof, wherein the means for containing said therapeutic dosages is selected from the group consisting of: the first and second agents are in a single dosage form; the first and second agents are packaged together in a single package or packette; the first and second agents are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device, and wherein the dosages are separated from each other within the pharmaceutical packaging system.

The invention provides a process for making a pharmaceutical composition of the invention comprising combining at least one GCR agonist or pharmaceutically acceptable salts thereof, at least one GCR antagonist or pharmaceutically acceptable salts thereof, and at least one pharmaceutically acceptable carrier.

The invention provides a method of treating a GC-responsive condition in a patient, comprising: administering a composition comprising: i) a first therapeutic agent which is a GCR agonist, or pharmaceutically acceptable salts thereof; ii) a second therapeutic agent which is a GCR antagonist or pharmaceutically acceptable salts thereof; and iii) at least one a pharmaceutically acceptable carrier, wherein the GCR agonist and the GCR antagonist are each present in an amount which, in combination, is a therapeutically effective amount for treating the GC-responsive condition in a patient.

The invention further provides a method of the invention wherein the amount of the GCR antagonist is sufficient to reduce a side-effect of administration of the GCR agonist.

The invention further provides a method of the invention wherein the GC-responsive condition include inflammatory conditions of the respiratory system; inflammatory conditions of the skin; musculo-skeletal system including bones, joints, connective tissue and muscle; gastrointestinal system including esophagus, intestines, mouth, salivary glands, stomach, liver, gallbladder, pancreas, rectum, and anus; circulatory system including blood vessels and heart; lymphatic system including lymph vessels and nodes; endocrine system; urinary system including kidneys, bladder, urethra and ureters; central and/or peripheral nervous system; and sensory organs.

The invention further provides a method of the invention wherein the side-effect of administration of the GCR agonist is selected from the group consisting of difficulty sleeping; feeling of a whirling motion; increased appetite; increased sweating; indigestion; mood changes; nervousness, blurring of vision; increased pressure in the eye, anaphylactoid reaction, anaphylaxis, angioedema, bradycardia, cardiac arrest, cardiac arrhythmias, cardiac enlargement, circulatory collapse, congestive heart failure, fat embolism, hypertension, hypertrophic cardiomyopathy in premature infants, myocardial rupture following recent myocardial infarction, edema, pulmonary edema, syncope, tachycardia, thromboembolism, thrombophlebitis, vasculitis, Acne, allergic dermatitis, dry scaly skin, ecchymoses and petechiae, erythema, impaired wound healing, increased sweating, rash, striae, suppression of reactions to skin tests, thin fragile skin, thinning scalp hair, urticarial, decreased carbohydrate and glucose tolerance, development of cushingoid state, hyperglycemia, glycosuria, hirsutism, hypertrichosis, increased requirements for insulin or oral hypoglycemic agents in diabetes, manifestations of latent diabetes mellitus, menstrual irregularities, secondary adrenocortical and pituitary unresponsiveness (particularly in times of stress, as in trauma, surgery, or illness), suppression of growth in pediatric patients, congestive heart failure in susceptible patients, fluid retention, hypokalemic alkalosis, potassium loss, sodium retention, abdominal distention, elevation in serum liver enzyme levels (usually reversible upon discontinuation), hepatomegaly, increased appetite, nausea, pancreatitis, peptic ulcer with possible perforation and hemorrhage, perforation of the small and large bowel (particularly in patients with inflammatory bowel disease), ulcerative esophagitis, negative nitrogen balance due to protein catabolism, aseptic necrosis of femoral and humeral heads, loss of muscle mass, muscle weakness, osteoporosis, pathologic fracture of long bones, steroid myopathy, tendon rupture, vertebral compression fractures, convulsions, depression, emotional instability, euphoria, headache, increased intracranial pressure with papilledema (pseudotumor cerebri) usually after treatment, insomnia, mood swings, neuritis, neuropathy, paresthesia, personality changes, psychic disorders, vertigo, exophthalmos, glaucoma, increased intraocular pressure, posterior subcapsular cataracts, abnormal fat deposits, decreased resistance to infection, hiccups, increased or decreased motility and number of spermatozoa, malaise, moon face, weight gain, and combinations thereof.

The invention further provides a method of the invention wherein the GCR agonist is selected from the group consisting of: alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, ulobetasol, and combinations thereof.

The invention further provides a method of the invention wherein the GCR antagonist is selected from the group consisting of ORG 34517, 11-(substituted phenyl)-estra-4,9-diene derivatives, and 11-(substituted phenyl)-estra-4,9-diene derivatives of formula I

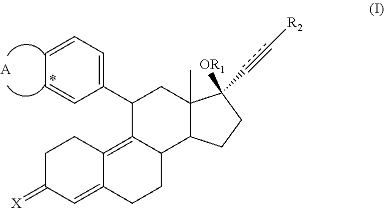

wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond.

The invention further provides a method of the invention wherein the GCR antagonist is naturally occurring.

The invention further provides a method of the invention wherein the GCR antagonist is is developed through chemical alterations of cholesterol or of physiologically normative steroid hormones.

The invention further provides a method of the invention wherein the GCR antagonist has a structure unrelated to cholesterol or other steroid hormones.

The invention provides a method of administration of a composition to a patient, comprising: administering to a patient a therapeutically effective amount of a composition for treating a GC-responsive condition, wherein the composition comprises: i) a first therapeutic agent which is a GCR agonist or pharmaceutically acceptable salt thereof; ii) a second therapeutic agent which is a GCR antagonist or pharmaceutically acceptable salts thereof; and iii) at least one pharmaceutically acceptable carrier, further wherein the GCR agonist and the GCR antagonist are each present in an amount which, in combination, is a therapeutically effective amount for treating a GC-responsive condition in a patient.

The invention further provides a method of the invention wherein the amount of the GCR antagonist is sufficient to reduce a side-effect of administration of the GCR agonist.

The invention further provides a method of the invention wherein the side-effect of administration of the GCR agonist is selected from the group consisting of difficulty sleeping; feeling of a whirling motion; increased appetite; increased sweating; indigestion; mood changes; nervousness, blurring of vision; increased pressure in the eye, anaphylactoid reaction, anaphylaxis, angioedema, bradycardia, cardiac arrest, cardiac arrhythmias, cardiac enlargement, circulatory collapse, congestive heart failure, fat embolism, hypertension, hypertrophic cardiomyopathy in premature infants, myocardial rupture following recent myocardial infarction, edema, pulmonary edema, syncope, tachycardia, thromboembolism, thrombophlebitis, vasculitis, Acne, allergic dermatitis, dry scaly skin, ecchymoses and petechiae, erythema, impaired wound healing, increased sweating, rash, striae, suppression of reactions to skin tests, thin fragile skin, thinning scalp hair, urticarial, decreased carbohydrate and glucose tolerance, development of cushingoid state, hyperglycemia, glycosuria, hirsutism, hypertrichosis, increased requirements for insulin or oral hypoglycemic agents in diabetes, manifestations of latent diabetes mellitus, menstrual irregularities, secondary adrenocortical and pituitary unresponsiveness (particularly in times of stress, as in trauma, surgery, or illness), suppression of growth in pediatric patients, congestive heart failure in susceptible patients, fluid retention, hypokalemic alkalosis, potassium loss, sodium retention, Abdominal distention, elevation in serum liver enzyme levels (usually reversible upon discontinuation), hepatomegaly, increased appetite, nausea, pancreatitis, peptic ulcer with possible perforation and hemorrhage, perforation of the small and large bowel (particularly in patients with inflammatory bowel disease), ulcerative esophagitis, negative nitrogen balance due to protein catabolism, aseptic necrosis of femoral and humeral heads, loss of muscle mass, muscle weakness, osteoporosis, pathologic fracture of long bones, steroid myopathy, tendon rupture, vertebral compression fractures, convulsions, depression, emotional instability, euphoria, headache, increased intracranial pressure with papilledema (pseudotumor cerebri) usually after treatment, insomnia, mood swings, neuritis, neuropathy, paresthesia, personality changes, psychic disorders, vertigo, exophthalmos, glaucoma, increased intraocular pressure, posterior subcapsular cataracts, abnormal fat deposits, decreased resistance to infection, hiccups, increased or decreased motility and number of spermatozoa, malaise, moon face, weight gain, and combinations thereof.

### DETAILED DESCRIPTION OF THE INVENTION

Compositions and methods for treating GC-responsive conditions and for reducing and preventing side-effects of GC treatment in a subject are provided by the present invention.

Methods of treating a GC-responsive condition in a subject are provided according to embodiments of the present invention which includes administering, in combination, a GCR agonist and a GCR antagonist in therapeutically effective amounts.

In particular embodiments, a method of treating a GC-responsive condition in a subject, is provided which includes administering, in combination, a therapeutically effective amount of a GCR agonist and a therapeutically effective amount of a GCR antagonist.

Methods of treating a GC-responsive condition in a subject are provided according to embodiments of the present invention which include administering in combination, a GCR agonist and a GCR antagonist in therapeutically effective amounts.

The phrase “administering in combination” as used herein refers to any form of administration of a GCR agonist and one or more GCR antagonists such that the GCR antagonist is administered to a subject while a previously administered GCR agonist is still effective in the subject or such that the GCR agonist is administered to a subject while a previously administered GCR antagonist is still effective in the subject.

The terms “treating” and “treatment” used to refer to treatment of a GC-responsive condition in a subject includes: preventing, inhibiting or ameliorating the GC-responsive condition in a subject, such as slowing progression of the condition and/or reducing or ameliorating a sign or symptom of the condition; and preventing, inhibiting or ameliorating a side-effect of GC administration GC-responsive condition in a subject. The terms “treating” and “treatment” are also used herein to refer to treatment of insulin resistance in a subject, such as GC-induced insulin resistance and insulin resistance resulting from factors such as high fat content diet, and include preventing, inhibiting or ameliorating insulin resistance in a subject or patient.

“Patient” for the purposes of the present invention includes humans and other animals, particularly mammals. Thus the compounds and methods are applicable to both human therapy and veterinary applications. In certain embodiments the subject is a mammal, and in a preferred embodiment the subject is human.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

Treatment of a GC-responsive condition with a combination of a GCR agonist and at least one GCR antagonist and a combination of two or more GCR antagonists allows for use of lower dosages of both the GCR agonist and the GCR antagonist to achieve a therapeutic effect than when either agonist is used alone. Thus, it is an aspect of the present invention that the amount of the GCR agonist used in a method of treating a GC-responsive condition is less than an amount of the GCR agonist necessary to achieve a therapeutic effect if administered in the absence of the GCR antagonist or combination of GCR antagonists.

In embodiments of the present invention, treatment of a GC-responsive condition with a combination of a GCR agonist and a GCR antagonist allows for use of lower dosages of both the GCR agonist and the GCR antagonist to achieve a therapeutic effect than when either agonist is used alone. Thus, it is an aspect of the present invention that the amount of the GCR agonist used in a method of treating a GC-responsive condition is less than an amount of the GCR agonist necessary to achieve a therapeutic effect if administered in the absence of the GCR antagonist.

In particular embodiments of the present invention, the amount of the GCR agonist administered is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90%, less than an amount of the GCR agonist necessary to achieve a therapeutic effect if administered without the GCR antagonist or combination of GCR antagonists. The amount of the GCR agonist administered can be less than 5% or more than 90%, less than an amount of the GCR agonist necessary to achieve a therapeutic effect if administered without the GCR antagonist or combination of GCR antagonists.

Side effects of GC treatment can be bothersome or even crippling. Side-effects of GCR agonists include, for example, osteoporosis, glaucoma, hyperglycemia, diabetes mellitus, sodium retention, hypertension, edematous face and other tissues, increased susceptibility to infection, decreased rate of wound healing, cataracts, acne, myopathy, joint pains, thinning of the skin or changes in skin color, redistribution of body fat to the nape of the neck and lower abdomen, suppression of the hypothalamic-pituitary-adrenal axis, euphoria, depression, emotional lability, psychoses, anxiety, anorexia, gastrointestinal ulceration, and hyperlipidemia, exaggerated sense of well-being, general body discomfort; headache, unusual weight gain, weakness, weight loss, symptoms of infection (e.g., fever, chills, sore throat), tendon or bone pain, unusual skin sensation, vision changes or other eye problems.

Methods of the present invention include administration of at least one GCR antagonist to prevent one or more GCR agonist side-effects. In particular embodiments, administration of one or more GCR antagonist reduces or prevents one or more GCR agonist side-effects.

In particular embodiments of the present invention, a GCR antagonist is administered to prevent or reduce hyperglycemia in a subject to whom a GCR agonist has been or will be administered.

In embodiments of methods of the present invention, a GCR agonist and a GCR antagonist are administered, in combination, to a subject having insulin resistance. Surprisingly, combined administration of a GCR agonist and a GCR antagonist prevents or reduces GC-induced side-effects such as hyperglycemia. Such methods are useful, for instance, in treating an insulin-resistant subject.

In particular embodiments of the present invention, GCR antagonist or combination of GCR antagonists is administered to prevent or reduce insulin resistance in a subject to whom a GCR agonist has been or will be administered. In a particular example, a GCR antagonist and a GCR agonist are administered in combination to prevent or reduce insulin resistance in a subject.

### GCR Antagonists

GCR antagonists bind to the receptor and prevent GCR agonists from binding and eliciting GR mediated events, including transcription. RU486 is an example of a non-selective GCR antagonist.

Compounds having high GCR binding affinity and, in addition, high in vivo anti-GC activity, while having, for example, low androgenic and progestagenic activities are disclosed in U.S. Pat. No. 6,011,025, incorporated herein by reference in its entirety. ORG 34517 is an example of a compound with high GCR binding affinity while having low androgenic and progestagenic activities.

It has been found that 11-(substituted phenyl)-estra-4,9-diene derivatives of formula I

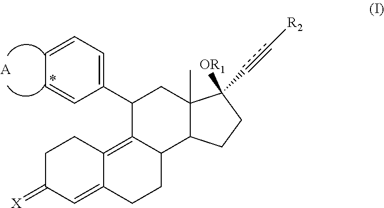

wherein
A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond, show specific and high GCR binding affinity and are highly active in vivo showing predominant anti-GC activity.

The compounds lack appreciable affinity for mineralocorticoid, progesterone, estrogen and androgen receptors, indicating a clean side effect profile.

The 11-(substituted phenyl)-estra-4,9-diene derivatives of the invention can be used in the prevention and treatment of GC dependent diseases or symptoms, like Cushing syndrome, diabetes, glaucoma, sleep disturbances, depression, anxiety, atherosclerosis, hypertension, adiposity, osteoporosis and withdrawal symptoms from narcotics and their mixtures.

Preferred compounds according to this invention are 11-(substituted phenyl)estra-4,9-diene derivatives, wherein the heteroatom(s) are (is) O, the 5- or 6-membered ring being optionally substituted with one or more fluorine atoms; R1 is H; and X is O or NOH.

More preferred compounds are 11-(substituted phenyl)estra-4,9-diene derivatives wherein A is a residue of a 5-membered ring. Particularly preferred are 11-(substituted phenyl)estra-4,9-diene derivatives wherein A contains 2 heteroatoms being O.

Especially preferred are 11-(substituted phenyl)estra-4,9-diene derivatives wherein R2 is methyl and the interrupted line represents a bond.

The most preferred compound is (11β,17β)-11-(1,3-benzodioxol-5-yl)-17-hydroxy-17-(1-propynyl)estra-4,9-dien-3-one (ORG 34517).

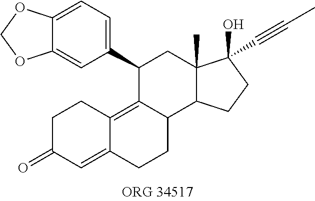

The term halogen means a fluorine, chlorine, bromine or iodine atom. Fluorine is the preferred halogen in ring A and when R2 is halogen, chlorine is preferred.
The terms (1-4C)alkyl and (1-8C)alkyl, as used in the definitions of R1 and R2, respectively, mean alkyl groups having 1-4 and 1-8 carbon atoms, respectively, for example methyl, ethyl, propyl, isopropyl, butyl, sec-butyl, tert-butyl, pentyl, neopentyl, hexyl, octyl.

The 11-(substituted phenyl)-estra-4,9-diene derivatives according to the present invention can be prepared by a process wherein a compound of formula II

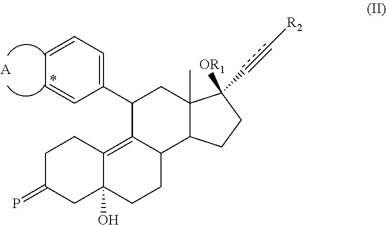

wherein A, R2 and the interrupted line have the meanings as previously defined, R1 is H, and P is a protected keto-group, is dehydrated and deprotected, after which the 1713-OH is optionally esterified by reaction with an appropriate carboxylic acid to give a derivative wherein R1 is 1-oxo(1-4C)alkyl, and optionally the 3-oxo group is converted into the corresponding 3-hydroxy- or 3-oxime derivative. The 3-oxo group can be reduced to form the 3-hydroxy-derivative by using a suitable reducing agent, such as sodium borohydride. The 3-oxime derivatives can be prepared by hydroxylamine treatment in a suitable solvent, like pyridine.

The derivatives of formula II may be prepared according to well known methods described and used for the preparation of steroids.

A suitable process for the preparation of derivatives of formula II starts from estra-4,9-diene-3,17-dione. Selective reduction of the 17-keto group to 17β-OH, 17α-H, e.g., with sodium borohydride, followed by protection of the 3-keto group, e.g., by ketalisation with ethyleneglycol, triethylorthoformate and p-toluenesulfonic acid, and oxidation of the 17-hydroxy group, e.g., with pyridinium chlorochromate, provides the 3-ketoprotected estra-5(10),9(11)-diene-3,17-dione. Alkynylation at the 17-position (yielding a 17α-alkynyl,17β-OH derivative), followed by epoxidation of the 5(10) double bond, e.g., with hydrogen peroxide, trifluoroacetophenone, and pyridine in dichloromethane according to the method as disclosed in European patent application EP 0 298 020, provides the 3-ketoprotected 5α,10α-epoxy-17α-alkynyl-17β-hydroxy-estr-9(11)-ene-3-one.

Subsequently, compounds of formula II are formed from this epoxide derivative, for example by reaction with an organometallic compound of the formula

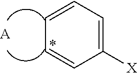

wherein X is a (alkali)metal, like lithium, or a magnesium halide, preferably magnesium bromide.

Suitable protective groups and methods to remove these groups are known in the art, for example, from T. W. Green: Protective Groups in Organic Synthesis (Wiley, NY, 1981). Particularly suitable protective groups for the protection of keto groups are acetals, e.g., 1,2-ethylene ketal.

The present invention relates to the fixed dose combination of a GR agonist with a tissue selective GR antagonist.

The specificity of ORG 34517 for GR blockade, without significant cross-binding to other related steroidal hormone receptors (such as those for estrogen and progesterone), eliminates the likelihood of significant toxicities and side effects. Indeed, none were identified in all the substantial phase I and phase II clinical trials that already have been performed with the compound. Because the drug is envisioned as being used in limited dosing over time, coordinated with the intermittent dosing strategies typical for chemotherapeutic agents, the GR blockade also would not lead to significant alteration of HPA-axis functioning, with rapid restitution of the HPA-axis to baseline following dosing.

### GCR Agonists

In certain embodiments, a GC may be employed in a method, composition, or kit of the invention. Suitable corticosteroids include, but are not limited to, those from the class of selective glucocorticosteroid receptor agonists (SEGRAs), such as, 11-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort; 21-deoxycortisol; dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin. Pharmaceutically acceptable salts, solvates and/or prodrugs of GCR agonists can be used. Combinations of two or more GCR agonists are contemplated as within the scope of the present invention.

The terms “pharmaceutically acceptable salt,” “pharmaceutically acceptable solvate” and “pharmaceutically acceptable prodrug” refers to salts, solvates and/or prodrugs which are suitable for use in a subject without undue toxicity or irritation to the subject and which are effective for their intended use.

Pharmaceutically acceptable salts include pharmaceutically acceptable acid addition salts and base addition salts. Pharmaceutically acceptable salts are well-known in the art, such as those detailed in S. M. Berge et al., J. Pharm. Sci., 66:1-19, 1977. Exemplary pharmaceutically acceptable salts are those suitable for use in a subject without undue toxicity or irritation to the subject and which are effective for their intended use which are formed with inorganic acids such as hydrochloric acid, hydrobromic acid, hydroiodic acid, nitric acid, phosphoric acid, sulfuric acid and sulfamic acid; organic acids such as acetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, formic acid, fumaric acid, glutamic acid, glycolic acid, glycerophosphoric acid, hemisulfic acid, heptanoic acid, hexanoic acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, trichloroacetic acid, trifluoroacetic acid and undecanoic acid; inorganic bases such as ammonia, hydroxide, carbonate, and bicarbonate of ammonium; organic bases such as Primary, secondary, tertiary and quaternary amine compounds ammonium, arginine, betaine, choline, caffeine, diolamine, diethylamine, diethanolamine, 2-dimethylaminoethanol, 2-diethylaminoethanol, dicyclohexylamine, dicyclohexylamine, dibenzylamine, N,N-dibenzylphenethylamine, 1-ephenamine, N,N′-dibenzylethylenediamine, ethanolamine, ethylamine, ethylenediamine, glucosamine, histidine, hydrabamine, isopropylamine, 1 h-imidazole, lysine, methylamine, N-ethylpiperidine, N-methylpiperidine, N-methylmorpholine, N,N-dimethylaniline, piperazine, trolamine, methylglucamine, purines, piperidine, pyridine, theobromine, tetramethylammonium compounds, tetraethylammonium compounds, trimethylamine, triethylamine, tripropylamine and tributylamine and metal cations such as aluminum, calcium, copper, iron, lithium, magnesium, manganese, potassium, sodium, and zinc.

Solvates illustratively include hydrates, ethanolates, methanolates.

### GC Responsive Conditions

The term “GC-responsive condition” refers to any disease or condition for which administration of one or more GCs has a beneficial effect. GC-responsive conditions that can be treated using compositions and methods of the present invention include, but are not limited to, inflammatory conditions and proliferative disorders.

GC-responsive conditions are well-known and include GC-responsive systemic and localized conditions such as GC-responsive conditions involving the upper airway passages, lower airway passages and/or lungs; skin; musculo-skeletal system including bones, joints, connective tissue and muscle; gastrointestinal system including esophagus, intestines, mouth, salivary glands, stomach, liver, gallbladder, pancreas, rectum, and anus; circulatory system including blood vessels and heart; lymphatic system including lymph vessels and nodes; endocrine system; urinary system including kidneys, bladder, urethra and ureters; central and/or peripheral nervous system; and sensory organs.

Exemplary GC-responsive conditions involving the upper airway passages, lower airway passages and/or lungs are adult respiratory distress syndrome, bronchiectasis, bronchial asthma, bronchitis, cystic fibrosis, pulmonary fibrosis, pulmonary inflammation, chronic obstructive pulmonary disease, edema, granulomatosis and sarcoidosis.

Exemplary GC-responsive conditions involving the skin are acne vulgaris, acne rosacea conglobata, acne rosacea fulminans, allergic uticaria, atopic dermatitis, eczema, psoriasis, pityriasis rubra pilaris, erythematous conditions, bullous dermatoses, epidermolysis bullosa, iethyoses, lichen planus, lichen simplex chronicus, lichenoid purpura, lichen sclerosus, pruritus, seborrheic dermatitis, rosacea, pemphigus vulgaris, erythema multiforme exudativum; alopecia greata, alopecia totalis, scarring, keloids, cutaneous sarcoidosis, pemphigoid gestationis, pemphigus vulgaris, wounds, burns, blisters, and cutaneous T cell lymphomas.

Exemplary GC-responsive conditions involving the musculo-skeletal system such as bones, joints, connective tissue and/or muscle are dermatomyositis, arthritic conditions generally, idiopathic arthritis; rheumatic diseases such as rheumatoid arthritis, juvenile rheumatoid arthritis; acute rheumatic fever, and polymyalgia rheumatica; rheumatoid spondylitis, gouty arthritis, osteoarthritis, polymyositis, systemic lupus erythematosus, scleroderma, Sjogren syndrome and Still disease.

Exemplary GC-responsive conditions involving the digestive system are biliary atresia, autoimmune or drug/toxin induced hepatitis, acute and chronic autoimmune or drug/toxin mediated pancreatitis, Crohn's disease, distal proctitis, gastritis, gastroenteritis, hemorrhoids, idiopathic proctitis, inflammatory bowel disease, sclerosing cholangitis and ulcerative colitis.

Exemplary GC-responsive conditions involving the circulatory system are atherosclerosis, Churg-Strauss syndrome, giant cell arteritis, Kawasaki disease, hypersensitivity vasculitis, mycocarditis, microscopic polyangiitis, polyarteritis nodosa, rheumatic carditis, Takayasu's arteritis, vasculitis and Wegener's granulomatosis.

Exemplary GC-responsive conditions involving the lymphatic system are histiocytic necrotizing lymphadenitis and proliferative diseases involving lymph nodes.

Exemplary GC-responsive conditions involving the endocrine system are thyroiditis, and deficiencies, such as, Addison's disease and adrenocortical insufficiency.

Exemplary GC-responsive conditions involving the urinary system are lupus nephritis, nephrotic syndrome, post-obstructive syndrome, tubular ischemia, and glomerulonephritides.

Exemplary GC-responsive conditions involving the nervous system are Bell's palsy, edema, multiple sclerosis and sequelae of ischemia (stroke).

Exemplary GC-responsive conditions involving the sensory organs are chorioretinitis, conjunctivitis, iritis, keratoconjunctivitis sicca, scleritis, uveitis, and macular edema.

GC-responsive inflammatory conditions are well-known and include systemic inflammatory conditions as well as organ, tissue or system-specific inflammatory conditions.

GC-responsive inflammatory conditions include autoimmune diseases such as rheumatoid arthritis, systemic lupus erythematosus, autoimmune hemolytic anemia; autoimmune hepatitis; Guillain-Barre syndrome and inflammatory bowel disease.

GC-responsive proliferative conditions illustratively include acute lymphatic leukemia; chronic lymphocytic leukemia; malignant lymphoma; lymphogranulomatosis; lymphosarcoma; and multiple myeloma.

GC-responsive conditions include tissue and organ transplant rejection and graft-versus-host disease.

GC-responsive conditions include blood disorders illustratively including acquired hemolytic anemia; non-hemolytic anemia, granulocytopenia, and idiopathic thrombocytopenia.

GC-responsive conditions include deficiencies such as Addison's disease and adrenocortical insufficiency.

Compositions and methods of the present invention are applicable to any condition having an inflammatory component and are not intended to be limited to use in conditions described herein.

For use in methods of the present invention, a GCR agonist and/or at least one GCR antagonist can be administered per se or with a pharmaceutically acceptable carrier.

### Side Effects

Side-effects of GCR agonists may include, for example, osteoporosis, glaucoma, hyperglycemia, diabetes mellitus, sodium retention, hypertension, edematous face and other tissues, increased susceptibility to infection, decreased rate of wound healing, cataracts, acne, myopathy, thinning of the skin, redistribution of body fat to the nape of the neck and lower abdomen, suppression of the hypothalamic-pituitary-adrenal axis, euphoria, depression, psychoses, anorexia, colonic ulceration, and hyperlipidemia.

Further, side-effects of GCR agonists may include, for example, difficulty sleeping; feeling of a whirling motion; increased appetite; increased sweating; indigestion; mood changes; nervousness, blurring of vision; increased pressure in the eye. Burning or stinging when you first put the medicine in your eye; dry, flaky skin; irritation; itching; redness; swelling; acne; clumsiness; dizziness; facial flushing; feeling of whirling motion; general body discomfort; headache; increased appetite; increased sweating; nausea; nervousness; pain, swelling, or redness at the injection site; sleeplessness; upset stomach; Severe allergic reactions (rash; hives; itching; difficulty breathing; tightness in the chest; swelling of the mouth, face, lips, or tongue); appetite loss; black, tarry stools; changes in menstrual periods; convulsions; depression; diarrhea; dizziness; exaggerated sense of well-being; fever; general body discomfort; headache; increased pressure in the eye; joint or muscle pain; mood swings; muscle weakness; personality changes; prolonged sore throat, cold, or fever; puffing of the face; severe nausea or vomiting; swelling of feet or legs; unusual weight gain; vomiting material that looks like coffee grounds; weakness; weight loss; changes in body fat; changes in menstrual periods; changes in skin color; chest pain; easy bruising or bleeding; mental or mood changes (e.g., depression); muscle pain, weakness, or wasting; swelling of feet or legs; seizures; severe nausea or vomiting; sudden severe dizziness or headache; symptoms of infection (eg, fever, chills, sore throat); tendon or bone pain; thinning of the skin; unusual skin sensation; unusual weight gain; vision changes or other eye problems; cataracts; changes in vision; continued or worsening itching or swelling; continuing blurred vision; discharge from eyes; eye pain; glaucoma; vision problems.

In addition, the following adverse reactions have been reported with dexamethasone or other corticosteroids: allergic reactions: anaphylactoid reaction, anaphylaxis, angioedema; cardiovascular: bradycardia, cardiac arrest, cardiac arrhythmias, cardiac enlargement, circulatory collapse, congestive heart failure, fat embolism, hypertension, hypertrophic cardiomyopathy in premature infants, myocardial rupture following recent myocardial infarction, edema, pulmonary edema, syncope, tachycardia, thromboembolism, thrombophlebitis, vasculitis; Dermatologic: Acne, allergic dermatitis, dry scaly skin, ecchymoses and petechiae, erythema, impaired wound healing, increased sweating, rash, striae, suppression of reactions to skin tests, thin fragile skin, thinning scalp hair, urticarial; endocrine: decreased carbohydrate and glucose tolerance, development of cushingoid state, hyperglycemia, glycosuria, hirsutism, hypertrichosis, increased requirements for insulin or oral hypoglycemic agents in diabetes, manifestations of latent diabetes mellitus, menstrual irregularities, secondary adrenocortical and pituitary unresponsiveness (particularly in times of stress, as in trauma, surgery, or illness), suppression of growth in pediatric patients; fluid and electrolyte disturbances: congestive heart failure in susceptible patients, fluid retention, hypokalemic alkalosis, potassium loss, sodium retention; gastrointestinal: abdominal distention, elevation in serum liver enzyme levels (usually reversible upon discontinuation), hepatomegaly, increased appetite, nausea, pancreatitis, peptic ulcer with possible perforation and hemorrhage, perforation of the small and large bowel (particularly in patients with inflammatory bowel disease), ulcerative esophagitis; metabolic: negative nitrogen balance due to protein catabolism; musculoskeletal: Aseptic necrosis of femoral and humeral heads, loss of muscle mass, muscle weakness, osteoporosis, pathologic fracture of long bones, steroid myopathy, tendon rupture, vertebral compression fractures; neurological/psychiatric: convulsions, depression, emotional instability, euphoria, headache, increased intracranial pressure with papilledema (pseudotumor cerebri) usually after treatment, insomnia, mood swings, neuritis, neuropathy, paresthesia, personality changes, psychic disorders, vertigo; ophthalmic: exophthalmos, glaucoma, increased intraocular pressure, posterior subcapsular cataracts; other: Abnormal fat deposits, decreased resistance to infection, hiccups, increased or decreased motility and number of spermatozoa, malaise, moon face, weight gain.

### Microarray Studies

Micro-array studies have delivered gene signatures from human CD4+ and CD14+ blood cells treated with prednisolone (Toonen et al. Pharmacogenomics, ‘in-press’). These gene signatures consist of both genes that are induced (through transactivation (TA)) and genes that are repressed (through transrepression (TR)). A classical GR antagonist—per definition—should antagonize all effects (both TR and TA) of prednisolone. Micro-array studies can be used to identify compounds or compound combinations that only induce the transrepression-effects of prednisolone without inducing the effects on transactivation. Thus, micro-array studies can be used to identify putative ‘partial antagonists’ or ‘selective antagonists’ that selectively inhibit only part of the (side) effect of prednisolone. Micro-array studies can also be done on tissue/material like for instance human whole blood that is treated with compounds in vitro.

It is hypothesized in the field that a GC agonistic action with a profile describing a transrepression/transactivation (TR/TA) ratio that is higher than that of prednisolone will show an insulin-sparing profile compared to prednisolone (less side effects).

The present invention claims a compound or compound-combination that mimics the transrepression profile of prednisolone without inducing GR related side effects. This compound or compound combination will be a safe and effective immune modulator.

### Formulations

The pharmaceutical compositions may be for human or animal usage in human and veterinary medicine and will typically comprise any one or more of a pharmaceutically acceptable diluent, carrier, or excipient.

The compounds of the invention may be administered enterally or parenterally, and for humans preferably in a daily dosage of 0.001-100 mg per kg body weight, preferably 0.01-10 mg per kg body weight. Mixed with pharmaceutically suitable auxiliaries, e.g., as described in the standard reference, Gennaro et al., Remington's Pharmaceutical Sciences, (18th ed., Mack Publishing Company, 1990, see especially Part 8: Pharmaceutical Preparations and Their Manufacture) the compounds may be compressed into solid dosage units, such as pills, tablets, or be processed into capsules or suppositories. By means of pharmaceutically suitable liquids, including nanosuspensions, the compounds can also be applied in the form of a solution, suspension, emulsion, e.g., for use as an injection preparation, for rectal or IV administration or eye drops, or as a spray, e.g., for use as a nasal spray, or formulated in a patch, with or without the addition of penetration enhancers, for transdermal delivery.

For making dosage units, e.g., tablets, the use of conventional additives such as fillers, colorants, polymeric binders and the like is contemplated. In general any pharmaceutically acceptable additive which does not interfere with the function of the active compounds can be used. Suitable carriers with which the compositions can be administered include lactose, starch, cellulose derivatives and the like, or mixtures thereof, used in suitable amounts.

The compounds of the invention may be administered enterally or parenterally. Mixed with pharmaceutically suitable auxiliaries, e.g., as described in the standard reference, Gennaro et al., Remington's Pharmaceutical Sciences. The compounds may be compressed into solid dosage units, such as pills, tablets, or be processed into capsules or suppositories. By means of pharmaceutically suitable liquids the compounds can also be applied in the form of a solution, suspension, emulsion, e.g., for use as an injection preparation or eye drops, or as a spray, e.g., for use as a nasal spray.

For making dosage units, e.g., tablets, the use of conventional additives such as fillers, colorants, polymeric binders and the like is contemplated. In general, any pharmaceutically acceptable additive which does not interfere with the function of the active compounds can be used. Suitable carriers with which the compositions can be administered include lactose, starch, cellulose derivatives and the like, or mixtures thereof, used in suitable amounts.

### Dosage Forms

The compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art. The compositions can be provided in the form of a mini-capsule, a capsule, a tablet, an implant, a troche, a lozenge (mini-tablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, ocular, urethral, transmucosal, vaginal, topical or rectal delivery.

The pharmaceutical composition can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be affected through favorable diffusion, dissolution, erosion, ion-exchange, osmosis or combinations thereof.

When formulated as a capsule, the capsule can be a hard or soft gelatin capsule, a starch capsule, or a cellulosic capsule. Although not limited to capsules, such dosage forms can further be coated with, for example, a seal coating, an enteric coating, an extended release coating, or a targeted delayed release coating. These various coatings are known in the art, but for clarity, the following brief descriptions are provided: seal coating, or coating with isolation layers: thin layers of up to 20 microns in thickness can be applied for variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are preferred for this application. HPMC and ethyl cellulose in combination, or Eudragit E100, may be particularly suitable for taste masking applications. Traditional enteric coating materials listed elsewhere can also be applied to form an isolating layer.

Extended release coatings are designed to effect delivery over an extended period of time. The extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness.

Enteric coatings are mixtures of pharmaceutically acceptable excipients which are applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Dosage forms of the compositions of the present invention can also be formulated as enteric coated delayed release oral dosage forms, i.e., as an oral dosage form of a pharmaceutical composition as described herein which utilizes an enteric coating to affect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

Delayed release generally refers to the delivery so that the release can be accomplished at some generally predictable location in the lower intestinal tract more distal to that which would have been accomplished if there had been no delayed release alterations. The preferred method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers for use in the present invention are anionic carboxylic polymers.

Shellac, also called purified lac, a refined product obtained from the, resinous secretion of an insect. This coating dissolves in media of pH>7.

Colorants, de-tackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method of the present invention, the combination of the invention may be administered to mammalian species, such as dogs, cats, humans, etc. and as such may be incorporated in a conventional systemic dosage form, such as a tablet, capsule, elixir or injectable. The above dosage forms will also include the necessary carrier material, excipient, lubricant, buffer, anti-bacterial, bulking agent (such as mannitol), anti-oxidants (ascorbic acid of sodium bisulfite) or the like.

The dose administered must be carefully adjusted according to age, weight and condition of the patient, as well as the route of administration, dosage form and regimen and the desired result.

The pharmaceutical compositions of the invention may be administered in the dosage forms in single or divided doses of one to four times daily. It may be advisable to start a patient on a low dose combination and work up gradually to a high dose combination.

Tablets of various sizes can be prepared, e.g., of about 1 to 2000 mg in total weight, containing one or both of the active pharmaceutical ingredients, with the remainder being a physiologically acceptable carrier of other materials according to accepted pharmaceutical practice. These tablets can be scored to provide for fractional doses. Gelatin capsules can be similarly formulated.

Liquid formulations can also be prepared by dissolving or suspending one or the combination of active substances in a conventional liquid vehicle acceptable for pharmaceutical administration so as to provide the desired dosage in one to four teaspoonful.

Dosage forms can be administered to the patient on a regimen of, for example, one, two, three, four, five, six, or other doses per day

In order to more finely regulate the dosage schedule, the active substances may be administered separately in individual dosage units at the same time or carefully coordinated times. Since blood levels are built up and maintained by a regulated schedule of administration, the same result is achieved by the simultaneous presence of the two substances. The respective substances can be individually formulated in separate unit dosage forms in a manner similar to that described above.

In formulating the compositions, the active substances, in the amounts described above, may be compounded according to accepted pharmaceutical practice with a physiologically acceptable vehicle, carrier, excipient, binder, preservative, stabilizer, flavor, etc., in the particular type of unit dosage form.

Illustrative of the adjuvants which may be incorporated in tablets are the following: a binder such as gum tragacanth, acacia, corn starch or gelatin; an excipient such as dicalcium phosphate or cellulose; a disintegrating agent such as corn starch, potato starch, alginic acid or the like; a lubricant such as stearic acid or magnesium stearate; a sweetening agent such as sucrose, aspartame, lactose or saccharin; a flavoring agent such as orange, peppermint, oil of wintergreen or cherry. When the dosage unit form is a capsule, it may contain in addition to materials of the above type a liquid carrier such as a fatty oil. Various other materials may be present as coatings or to otherwise modify the physical form of the dosage unit. For instance, tablets or capsules may be coated with shellac, sugars (including those other than D-Glucose or Dextrose) or both. A syrup of elixir may contain the active compound, water, alcohol or the like as the carrier, glycerol as solubilizer, sucrose as sweetening agent, methyl and propyl parabens as preservatives, a dye and a flavoring such as cherry or orange.

One embodiment of this invention includes methods of treating, preventing, or diagnosing a particular disease or condition by administering the disclosed nanoparticles, composite nanoparticles, nanosuspension, or nanocapsules to a subject. In many instances, the nanoparticles, composite nanoparticles, or nanocapsules are administered alone or can be included within a pharmaceutical composition. An effective amount of a pharmaceutical composition, generally, is defined as that amount sufficient to ameliorate, reduce, minimize, or limit the extent of the disease or condition. More rigorous definitions may apply, including elimination, eradication, or cure of the disease or condition.

“Nanoparticles” are solid particles of an average particle diameter of, for example, less than about 1 micron (micrometer). One micron is 1,000 nanometers (nm).

“Stabilized” nanoparticles are nanoparticles coated with a stabilizing material and having a reduced tendency for aggregation and loss of dispersion with respect to nanoparticles of the compound of the invention without a stabilizing coating.

A nano-spray is a spray containing nano-particles or a spray that produces nano-particles. A nano-dispersion is a dispersion containing nanoparticles. A nano-suspension is a suspension containing nano-particles.

The liquid formulations useful herein may comprise a solvent, solution, suspension, micro-suspension, nano-suspension, emulsion, micro-emulsion, gel or even a melt containing the active component or components. In some embodiments the nano-particles, nano-fibers, or nano-fibrils may be in the form of, or within or on, granules, powders, suspensions, solutions, dissolvable films, mats, webs, tablets, or releasable forms particularly releasable dosage forms. Other particular useful forms are concentrates to which a diluting liquid is added prior to use. The product may also be sprayed onto the inner surface of a container to which a liquid is added later prior to use and the nano-particles, nano-fibers, or nano-fibrils, are released into the liquid.

Pharmaceutical compositions of the present invention can include nano-particles, composite nano-particles, nano-suspension, or nano-capsules of the present invention.

In certain non-limiting embodiments, pharmaceutical compositions may comprise, for example, at least about 0.1% of an active ingredient or nano-particles, composite nano-particles, or nano-capsules, for example. In other embodiments, the active ingredient or nano-particles, composite nano-particles, or nano-capsules may comprise between about 2% to about 75% of the weight of the unit, or between about 25% to about 60%, for example, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered.

The composition may also include various antioxidants to retard oxidation of one or more active ingredient or nano-particles, composite nano-particles, nano-suspension, or nano-capsules. The prevention of the action of microorganisms can be brought about by preservatives such as various anti-bacterial and anti-fungal agents, including but not limited to parabens (e.g., methylparabens, propylparabens), chlorobutanol, phenol, sorbic acid, or combinations thereof.

In order to increase the effectiveness of a treatment with the nano-particles, nano-gels, composite nano-particles, nano-suspension, or nano-capsules of the present invention, it may be desirable to combine these nano-particles, composite nano-particles, or nano-capsules with other therapies effective in the treatment of a particular disease or condition.

The formulations as described above may be administered for a prolonged period, that is, for as long as the potential for a disease or condition remains or the symptoms continue.

### Packaging/Treatment Kits

The present invention relates to a kit for conveniently and effectively carrying out the methods in accordance with the present invention. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging pharmaceutical unit dosage forms. If desired, the blister can be in the form of a childproof blister, i.e., a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as an AM dose is packaged with a “mid day” and a PM dose; or an AM dose is packaged with a PM dose. Alternatively, placebo dosages, or vitamin or dietary supplements, either in a form similar to or distinct from the pharmaceutical active dosages, can be included.

In one aspect, the package, kit or container comprises a “blister package” (also called a blister pack, or bubble pack). In one aspect, the blister package consists of two or more separate compartments: AM dosage of this invention, and PM dosage of this invention, or mid-day dosage of this invention. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can be comprised of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a pharmaceutical combination of the invention), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or large. Conventional blister packs can also be sealed (e.g., using an AERGO 8 DUO®, SCA Consumer Packaging, Inc., DeKalb, Ill.) using regular heat seal tooling. This alternative aspect, using heat seal tooling, can seal common types of thermoformed packaging.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, laminated aluminum foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminum (e.g., alufoil) lidding material. In one aspect, hermetically-sealed high barrier aluminum (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

The present invention can best be illustrated by the following prophetic example.

Prednisolone at a therapeutic dose induces both transrepression (light gray, wanted effects) and transactivation (dark gray, unwanted effects).

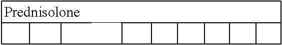

The ideal compound should only have transrepression effects

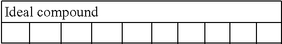

This can be achieved by:
1. partial agonists for the GR.
2. combining prednisolone with partial antagonists; compounds that selectively block the transactivation effects.

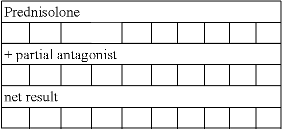

3. combining partial antagonists and agonists to create the transrepression effect of prednisolone

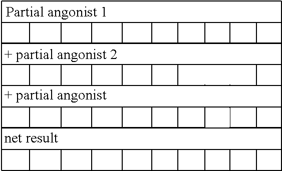

While the invention has been described in detail and with reference to specific examples thereof, it will be apparent to one skilled in the art that various changes and modifications can be made therein without departing from the spirit and scope thereof.

What is claimed is:

1. A composition, comprising:
i) a first therapeutic agent which is a GCR agonist or pharmaceutically acceptable salt thereof;
ii) a second therapeutic agent which is a GCR antagonist or pharmaceutically acceptable salt thereof; and
iii) at least one pharmaceutically acceptable carrier;
wherein the GCR agonist and the GCR antagonist are each present in an amount which, in combination, is a therapeutically effective amount for treating a GC-responsive condition in a patient.

2. The composition of claim 1, wherein the amount of the GCR antagonist is sufficient to reduce a side-effect of administration of the GCR agonist.

3. The composition of claim 1, wherein the GCR agonist is selected from the group consisting of: alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, ulobetasol, and combinations thereof.

4. The composition of claim 1, wherein the GCR antagonist is selected from the group consisting of ORG 34517, 11-(substituted phenyl)-estra-4,9-diene derivatives, and 11-(substituted phenyl)-estra-4,9-diene derivatives of formula I 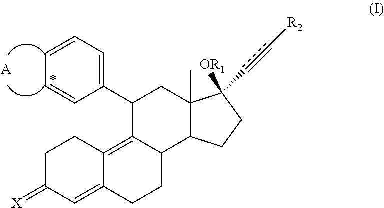
wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond.

5. The composition of claim 1, wherein the GCR antagonist is naturally occurring.

6. The composition of claim 1, wherein the GCR antagonist is developed through chemical alterations of cholesterol or of physiologically normative steroid hormones.

7. The composition of claim 1, wherein the GCR antagonist has a structure unrelated to cholesterol or other steroid hormones.

8. The composition of claim 1, wherein the composition is a pharmaceutical composition.

9. A pharmaceutical composition comprising:
i) a first therapeutic agent which is a GCR agonist or pharmaceutically acceptable salt thereof;
ii) a second therapeutic agent which is a GCR antagonist or pharmaceutically acceptable salt thereof; and
iii) at least one pharmaceutically acceptable carrier,
wherein the pharmaceutical composition is formulated or manufactured as a liquid, an elixir, an aerosol, a spray, a powder, a tablet, a pill, a capsule, a gel, a geltab, a nano-suspension, a nano-particle, an extended release dosage form, or a topical formulation, further wherein the GCR agonist and the GCR antagonist are each present in an amount which, in combination, is a therapeutically effective amount for treating a GC-responsive condition in a patient.

10. The pharmaceutical composition of claim 9, wherein the amount of the GCR antagonist is sufficient to reduce a side-effect of administration of the GCR agonist.

11. A combination therapy which comprises:
i) a first therapeutic agent which is a GCR agonist or pharmaceutically acceptable salt thereof;
ii) a second therapeutic agent which is a GCR antagonist or pharmaceutically acceptable salt thereof.

12. A pharmaceutical composition made by combining at least one GCR agonist or pharmaceutically acceptable salt thereof, at least one a GCR antagonist or pharmaceutically acceptable salt thereof, and a pharmaceutically acceptable carrier.

13. A pharmaceutical active substance combination comprising:
i) a first therapeutic agent which is a GCR agonist or pharmaceutically acceptable salt thereof;
ii) a second therapeutic agent which is a GCR antagonist or pharmaceutically acceptable salts thereof,
as a combination product for simultaneous, separate, or sequential use.

14. A pharmaceutical dosage form comprising:
i) a first therapeutic agent which is a GCR agonist or pharmaceutically acceptable salt thereof;
ii) a second therapeutic agent which is a GCR antagonist or pharmaceutically acceptable salt thereof, wherein the first and second agents are in multiple separated dosage units or in a single dosage unit of a combination of the therapeutic agents.

15. A kit for the treatment, amelioration or prevention of a GC-responsive condition in a patient in need of such treatment comprising:
(a) the pharmaceutical composition of claim 9; and
(b) at least one blister package; a lidded blister; a blister card or packet; a clamshell; an intravenous (IV) package, IV packette or IV container; a tray or a shrink wrap comprising the pharmaceutical composition of (a) and instructions for use of the pharmaceutical composition.

16. A product of manufacture comprising: a blister package; a lidded blister; a blister card or packet; a clamshell; an intravenous (IV) package, IV packette or IV container; a tray or a shrink wrap comprising the pharmaceutical composition of claim 9 and instructions for use of the pharmaceutical composition.

17. A pharmaceutical packaging system comprising:
i) a first therapeutic agent which is a GCR agonist, or pharmaceutically acceptable salts thereof;
ii) a second therapeutic agent which is a GCR antagonist, or pharmaceutically acceptable salts thereof,
wherein the means for containing said therapeutic dosages is selected from the group consisting of the first and second agents are in a single dosage form;
the first and second agents are packaged together in a single package or packette; the first and second agents are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device, and wherein the dosages are separated from each other within the pharmaceutical packaging system.

18. A process for making a pharmaceutical composition comprising combining at least one GCR agonist or pharmaceutically acceptable salts thereof, at least one GCR antagonist or pharmaceutically acceptable salts thereof, and at least one pharmaceutically acceptable carrier.

19. A method of treating a GC-responsive condition in a patient, comprising: administering a composition comprising:
i) a first therapeutic agent which is a GCR agonist, or pharmaceutically acceptable salts thereof;
ii) a second therapeutic agent which is a GCR antagonist or pharmaceutically acceptable salts thereof; and
iii) at least one a pharmaceutically acceptable carrier,
wherein the GCR agonist and the GCR antagonist are each present in an amount which, in combination, is a therapeutically effective amount for treating the GC-responsive condition in a patient.

20. The method of claim 19, wherein the amount of the GCR antagonist is sufficient to reduce a side-effect of administration of the GCR agonist.

21. The method of claim 19, wherein the GC-responsive condition is selected from the group consisting of inflammatory conditions of the respiratory system; inflammatory conditions of the skin; musculo-skeletal system including bones, joints, connective tissue and muscle; gastrointestinal system including esophagus, intestines, mouth, salivary glands, stomach, liver, gallbladder, pancreas, rectum, and anus; circulatory system including blood vessels and heart; lymphatic system including lymph vessels and nodes; endocrine system; urinary system including kidneys, bladder, urethra and ureters; central and/or peripheral nervous system; and sensory organs.

22. The method of claim 19, wherein the side-effect of administration of the GCR agonist is selected from the group consisting of difficulty sleeping; feeling of a whirling motion; increased appetite; increased sweating; indigestion; mood changes; nervousness, blurring of vision; increased pressure in the eye, anaphylactoid reaction, anaphylaxis, angioedema, bradycardia, cardiac arrest, cardiac arrhythmias, cardiac enlargement, circulatory collapse, congestive heart failure, fat embolism, hypertension, hypertrophic cardiomyopathy in premature infants, myocardial rupture following recent myocardial infarction, edema, pulmonary edema, syncope, tachycardia, thromboembolism, thrombophlebitis, vasculitis, Acne, allergic dermatitis, dry scaly skin, ecchymoses and petechiae, erythema, impaired wound healing, increased sweating, rash, striae, suppression of reactions to skin tests, thin fragile skin, thinning scalp hair, urticarial, decreased carbohydrate and glucose tolerance, development of cushingoid state, hyperglycemia, glycosuria, hirsutism, hypertrichosis, increased requirements for insulin or oral hypoglycemic agents in diabetes, manifestations of latent diabetes mellitus, menstrual irregularities, secondary adrenocortical and pituitary unresponsiveness (particularly in times of stress, as in trauma, surgery, or illness), suppression of growth in pediatric patients, congestive heart failure in susceptible patients, fluid retention, hypokalemic alkalosis, potassium loss, sodium retention, abdominal distention, elevation in serum liver enzyme levels (usually reversible upon discontinuation), hepatomegaly, increased appetite, nausea, pancreatitis, peptic ulcer with possible perforation and hemorrhage, perforation of the small and large bowel (particularly in patients with inflammatory bowel disease), ulcerative esophagitis, negative nitrogen balance due to protein catabolism, aseptic necrosis of femoral and humeral heads, loss of muscle mass, muscle weakness, osteoporosis, pathologic fracture of long bones, steroid myopathy, tendon rupture, vertebral compression fractures, convulsions, depression, emotional instability, euphoria, headache, increased intracranial pressure with papilledema (pseudotumor cerebri) usually after treatment, insomnia, mood swings, neuritis, neuropathy, paresthesia, personality changes, psychic disorders, vertigo, exophthalmos, glaucoma, increased intraocular pressure, posterior subcapsular cataracts, abnormal fat deposits, decreased resistance to infection, hiccups, increased or decreased motility and number of spermatozoa, malaise, moon face, weight gain, and combinations thereof.

23. The method of claim 19 wherein the GCR agonist is selected from the group consisting of: alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, ulobetasol, and combinations thereof.

24. The method of claim 19, wherein the GCR antagonist is selected from the group consisting of ORG 34517, 11-(substituted phenyl)-estra-4,9-diene derivatives, and 11-(substituted phenyl)-estra-4,9-diene derivatives of formula I 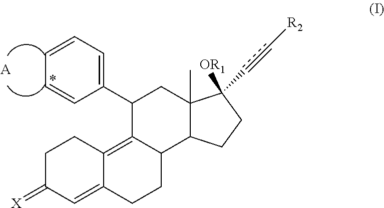
wherein A is a residue of a 5- or 6-membered ring containing 2 heteroatoms which are not connected to each other and independently selected from O and S, the ring being optionally substituted with one or more halogen atoms, or A is a residue of a 5- or 6-membered ring wherein no double C—C bonds are present, containing 1 heteroatom selected from O and S, which heteroatom is connected to the phenyl group at the position indicated with an asterisk, the ring being optionally substituted with one or more halogen atoms; R1 is H or 1-oxo(1-4C)alkyl; R2 is H, (1-8C)alkyl, halogen or CF3; X is selected from (H,OH), O, and NOH; and the interrupted line represents an optional bond.

25. The method of claim 19, wherein the GCR antagonist is naturally occurring.

26. The method of claim 19, wherein the GCR antagonist is developed through chemical alterations of cholesterol or of physiologically normative steroid hormones.

27. The method of claim 19, wherein the GCR antagonist has a structure unrelated to cholesterol or other steroid hormones.

28. A method of administration of a composition to a patient, comprising:
administering to a patient a therapeutically effective amount of a composition for treating a GC-responsive condition,
wherein the composition comprises:
i) a first therapeutic agent which is a GCR agonist or pharmaceutically acceptable salt thereof;
ii) a second therapeutic agent which is a GCR antagonist or pharmaceutically acceptable salts thereof; and
iii) at least one pharmaceutically acceptable carrier,
further wherein the GCR agonist and the GCR antagonist are each present in an amount which, in combination, is a therapeutically effective amount for treating a GC-responsive condition in a patient.

29. The method of claim 28, wherein the amount of the GCR antagonist is sufficient to reduce a side-effect of administration of the GCR agonist.

30. The method of claim 28, wherein the side-effect of administration of the GCR agonist is selected from the group consisting of difficulty sleeping; feeling of a whirling motion; increased appetite; increased sweating; indigestion; mood changes; nervousness, blurring of vision; increased pressure in the eye, anaphylactoid reaction, anaphylaxis, angioedema, bradycardia, cardiac arrest, cardiac arrhythmias, cardiac enlargement, circulatory collapse, congestive heart failure, fat embolism, hypertension, hypertrophic cardiomyopathy in premature infants, myocardial rupture following recent myocardial infarction, edema, pulmonary edema, syncope, tachycardia, thromboembolism, thrombophlebitis, vasculitis, Acne, allergic dermatitis, dry scaly skin, ecchymoses and petechiae, erythema, impaired wound healing, increased sweating, rash, striae, suppression of reactions to skin tests, thin fragile skin, thinning scalp hair, urticarial, decreased carbohydrate and glucose tolerance, development of cushingoid state, hyperglycemia, glycosuria, hirsutism, hypertrichosis, increased requirements for insulin or oral hypoglycemic agents in diabetes, manifestations of latent diabetes mellitus, menstrual irregularities, secondary adrenocortical and pituitary unresponsiveness (particularly in times of stress, as in trauma, surgery, or illness), suppression of growth in pediatric patients, congestive heart failure in susceptible patients, fluid retention, hypokalemic alkalosis, potassium loss, sodium retention, Abdominal distention, elevation in serum liver enzyme levels (usually reversible upon discontinuation), hepatomegaly, increased appetite, nausea, pancreatitis, peptic ulcer with possible perforation and hemorrhage, perforation of the small and large bowel (particularly in patients with inflammatory bowel disease), ulcerative esophagitis, negative nitrogen balance due to protein catabolism, aseptic necrosis of femoral and humeral heads, loss of muscle mass, muscle weakness, osteoporosis, pathologic fracture of long bones, steroid myopathy, tendon rupture, vertebral compression fractures, convulsions, depression, emotional instability, euphoria, headache, increased intracranial pressure with papilledema (pseudotumor cerebri) usually after treatment, insomnia, mood swings, neuritis, neuropathy, paresthesia, personality changes, psychic disorders, vertigo, exophthalmos, glaucoma, increased intraocular pressure, posterior subcapsular cataracts, abnormal fat deposits, decreased resistance to infection, hiccups, increased or decreased motility and number of spermatozoa, malaise, moon face, weight gain, and combinations thereof.
